(19) United States
(12) Patent Application Publication
(10) Pub. No.: US 2005/0027267 A1
(43) Pub. Date: Feb. 3, 2005

(54) Absorbent article with improved fit and free liquid intake

(76) Inventors: Wendy Lynn Van Dyke, Appleton, WI (US); Thomas Harold Roessler, Appleton, WI (US); Amber Marie Fortune, Kaukauna, WI (US); David Louis Zenker, Neenah, WI (US)

Correspondence Address: Roland W. Norris;Pauley Petersen Kinne & Erickson, Suite 365, 2800 West Higgins Road, Hoffman Estates, IL, 60195, US

(21) Appl. No.: 10/631,641

(22) Filed: Jul. 31, 2003

(51) Int. Cl. A61F 13/15; A61F 13/20

(52) U.S. Cl. 604/367

(57) ABSTRACT

A disposable absorbent diaper has an outer cover, a bodyside liner, and an absorbent core positioned between the outer cover and the bodyside liner. A lofty, resilient, liquid permeable lofty spacer layer is positioned between the absorbent core and the outer cover and in liquid communication with the absorbent core. The absorbent core can be made from standard airformed materials which may have a combination of reduced surface area, narrow crotch width and a permeability level which may permit free liquid run off during insult to the garment. The lofty spacer layer has a high void volume and is also made of economical materials and desirably has a width in the crotch region greater than a width of the absorbent core in the crotch region to provide distribution of the free liquid run off underneath the absorbent core, allowing for reduction of the width of the absorbent core in the crotch region to provide an economical diaper with improved fit and leakage while reducing reliance on a gasketing system. The lofty resilient spacer layer will desorb into the absorbent core between insults thereby retaining its spacer layer function for use with breathable outer covers.

### BACKGROUND OF THE INVENTION

Within the field of disposable absorbent article manufacture, e.g. diapers, there are three primary goals, a reasonable cost, comfort of the wearer, and avoidance of leakage of exudates beyond the boundary of the garment. Thus, typically a disposable diaper is designed to capture the exudates, or the free liquids thereof, as quickly as possible, i.e., essentially trying to allow no free liquid to exist. Typically, a surge layer is provided under the bodyside liner (liner) and over the absorbent core (core) of the garment at the target area of insult to quickly capture and distribute the free liquid to an absorbent core of liquid retention materials. Past suggestions for doing this regularly envision improvements to the surge or absorbent materials which often result in additional material costs.

Generally, it is believed that the known art has focused on utilizing elaborate materials and structures in an attempt to prevent any free liquid from existing within the garment. Thus, great emphasis has been placed on rapid acting surge layers, high uptake rate absorbent cores, and combinations of the two, which if implemented would give rise to more expensive absorbent products for the consumer.

If material improvements prove cost prohibitive, another way to help prevent leaks is to ensure that an adequate surface area of the garment is available for free liquid capture. This leads to a typical absorbent core (the most bulky component of the diaper) width in the crotch of most diapers of 3.5 inches or greater. However, there has long been a desire in the art to reduce the crotch width, desirably to a more comfortable 2.5 inches or less. But, loss of garment crotch area available for both free liquid distribution and retention material mass may increase the chance of leakage. Such a loss of liquid retention area or mass can be made up for by making the crotch area absorbent core of higher basis weight materials resulting in a thicker crotch, or more dense with retention materials, or both. However, these solutions may lead to lower permeability and longer liquid uptake times and the narrower crotch still lacks surface area available for the control of free liquids. Thus, an insult to the narrower garment will allow more free liquid to reach the edges of the garment faster.

Again therefore, prior thinking on disposable absorbent garment construction would dictate that to have a comfortable crotch width and avoid free liquids, one must use expensive materials thereby adding to the cost of garment construction. Accordingly, there remains a desire for an absorbent disposable article that has a narrow absorbent system in the crotch region to provide a comfortable fit without negatively effecting cost or leakage of the garment.

### SUMMARY OF THE INVENTION

In response to the difficulties and problems discussed above, in contrast to traditional thinking, a garment according to the present invention takes the novel approach of providing a narrow absorbent core in the crotch region desirably made from economical and known materials and processes and allowing free liquid to run off the absorbent structure (such liquid also referred to herein as “run off”) upon insult to the garment. A lofty spacer layer having a high void volume and high resilience is substituted for the thin tissue or nonwoven spacer layers typically inserted between the core and the outer cover. The lofty spacer layer of the present invention economically contains and distributes the run off before it leaks out the diaper. According to some aspects of the invention, the lofty, i.e., low density and high void volume, spacer layer may be a resilient, economical, material such as used for a typical surge layer between the liner and the core. The lofty spacer layer desirably has a width greater than the absorbent core in the crotch region and desirably may have a width substantially equal to the width of the garment crotch region but less than the total garment exterior width.

An absorbent system of a garment according to the present invention generally comprises, or may consist essentially of, an absorbent core performing the main liquid retention function and a lofty spacer layer between the liner and the outer cover. A fluid insult which has an amount, or rate, greater than the ability of the absorbent system to immediately capture the liquid will result in some run off, or free liquid. The run off can be captured by a lofty spacer layer, desirably wider than the core, which is in liquid communication with the absorbent core. The run off is then desorbed from the lofty spacer layer to the absorbent core rather than being free to travel along the outer cover of the garment. Further, according to some aspects of the invention, the lofty spacer layer performs well with breathable outer covers since it desorbs into the absorbent core and thereafter maintains a spacer function. Certain embodiments of the lofty spacer layer are extendible and therefore also perform well with extendible outer cover materials.

Unexpectedly, use of the lofty spacer according to the present invention provides a significant leakage benefit with economical airformed, lower permeability, absorbent cores. Further, the lofty spacer layer is believed to provide an improvement for higher permeability absorbent cores. The benefit of the lofty spacer layer is more apparent in lower permeability absorbent structures due to the higher incidence of run off thereby allowing the lofty spacer mechanism to work. This benefit is reduced in the higher permeability absorbents due to the reduced potential for run off. Another benefit is that by using a lofty spacer layer, an airformed absorbent core diaper can be made to perform as well as a more costly stabilized airlaid absorbent with a current diaper design, but at lower material cost, capital cost, higher efficiency, and reduced process complexity. An additional benefit is that by using a lofty spacer layer, an airformed core absorbent diaper can perform as well as a stabilized airlaid core absorbent diaper without the adverse effect on fit and comfort of reduced absorbent flexibility and conformability associated with stabilized airlaid material.

Narrower crotch absorbent cores provide better fit but often suffer from higher leakage as a result of reduced liquid intake functionality. Still another benefit of this invention is that, because of the increased liquid transfer efficiency of the present invention, a narrower crotch absorbent can perform as well as a wider crotch absorbent core and therefore provide improved fit without a tradeoff in leakage performance. The crotch region of a garment according to the present invention can thus be made narrower than the crotch region of a conventional garment to provide proper fit without sacrifice of leakage performance. The narrow absorbent core of the crotch area may be made from a higher basis weight and/or thickened material, as compared to a standard core, to prevent loss of required absorbent material mass. The run off will be captured efficiently by the lofty spacer layer of the present invention. The effective surface area of the liquid containment structures of the garment for free liquid absorption is thus placed to account for increased free liquid at the underside of the absorbent core.

Aspects of the present invention will include a lofty spacer layer positioned between the outer cover and the absorbent core and in liquid communication with a major surface of the absorbent core. The lofty spacer layer is positioned in the crotch region and may extend longitudinally into at least a portion of the front waist region and/or longitudinally into at least a portion of the back waist region. The lofty spacer layer desirably has a width in the crotch region greater than the width of the absorbent layer in the crotch region. Desirably, the lofty spacer layer has a width (generally parallel to the transverse axis) in the crotch region to extend at least about 0.25 inches beyond a corresponding or adjacent edge of the absorbent core in the crotch region, more desirably at least about 0.5 inches beyond.

The reduced width of the absorbent core in the crotch region of the garment provides improved fit while the porous, liquid permeable, high void volume, resilient, lofty spacer layer between the absorbent core and the outer cover provides a means for distributing the free liquid exudates to a major plane surface of the absorbent core to provide an increased surface area for free liquid absorption. For example, the absorbent core of the present invention may suitably have a width in the crotch region not greater than about 3 inches, desirably less than about 2.5 inches and in many cases less than about 2.0 inches for an infant diaper. According to some embodiments the core and lofty spacer layer combination may eliminate the need for a surge layer between the liner and the core, especially where higher basis weight lofty spacer layers are used.

### DEFINITIONS

“Absorbent core,” or “core,” is the main liquid absorption and retention layer of the absorbent garment.

“Airformed” is a process whereby absorbent materials are blown onto a forming drum or wire to form individual batts or matrices of unbonded absorbent material suitable for placement in a single garment.

“Airlaid” is a process including the blowing of absorbent materials including binder materials onto a large collecting form, such as a collecting wire, where the binder materials are then activated. The sheets of material are then cut to size for placement in individual garments.

As used herein, the term “bonded” refers to the joining, adhering, connecting, attaching, or the like, of two elements. Two elements will be considered bonded together when they are bonded directly to one another or indirectly to one another, such as when each is directly bonded to intermediate elements.

As used herein, the term “bonded-carded web” refers to webs that are made from staple fibers which are sent through a combing or carding unit, which separates or breaks apart and aligns the staple fibers in the machine direction to form a generally machine direction-oriented fibrous nonwoven web. Such fibers are usually purchased in bales, which are placed in an opener/blender or picker, which separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface if so desired. Another suitable and well-known bonding method, particularly when using bicomponent staple fibers, is through-air bonding.

As used herein, the term “comprises,” “comprising,” and other derivatives from the root term “comprise” are intended to be open-ended terms that specify the presence of any stated features, elements, integers, steps or components, but do not preclude the presence or addition of one or more other features, elements, integers, steps, components or groups thereof. Accordingly, such terms are intended to be synonymous with “has,” “have,” “having,” “includes,” “including,” and any derivatives of these words.

As used herein, the term “consisting essentially of” does not exclude the presence of additional materials which do not significantly affect the desired characteristics of a given composition or product.

As used herein, the term “disposable” refers to articles that are designed to be discarded after a limited use rather than being laundered or otherwise restored for reuse.

The terms “extendible,” “extensible,” and “expandable” are used interchangeably herein to mean a material which upon application of a stretching force, can be extended in a particular direction (e.g., the cross-direction), to a stretched dimension (e.g., width) which is at least 25% greater than an original, unstretched dimension. When the stretching force is removed after a one-minute holding period, the material preferably does not retract, or retracts by not more than 30%, of the difference between the stretched dimension and the original dimension.

As used herein, the term “fabrics” is used to refer to all of the woven, knitted and nonwoven fibrous webs.

As used herein, the term “integral” is used to refer to various portions of a single unitary element rather than separate structures bonded to or placed with or placed near one another.

As used herein, the term “layer” when used in the singular can have the dual meaning of a single element or a plurality of elements.

As used herein, the term “liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, may spread or be transported parallel to the plane of the liquid impermeable layer or laminate, but this is not considered to be within the meaning of “liquid impermeable” when used herein.

“Liquid communication” means a fluid transmissive relationship between at least two entities whereby the entities may transfer fluids from one of the entities to the other, or between the entities.

A “lofty” spacer layer material according to the present invention generally refers to a resilient nonwoven web having a basis weight of at least about 0.5 osy (17 grams per square meter (gsm)), a void volume of greater than 15 centimeters cubed (or cubic centimeters) per gram (cc/g) and a void volume per area of greater than 0.03 cc/cm², while under a pressure of 0.05 pounds per square inch (psi) (0.34 kilopascal). One type of fibrous material which can be made to these specifications is generally described in, and generally measured in accordance with the procedures of, U.S. Pat. No. 5,486,166 to Bishop, which is incorporated herein by reference in its entirety.

As used herein, the terms “longitudinal” and “transverse” or “lateral” have their customary meanings. The longitudinal axis lies in the plane of the article when laid flat and fully extended and is generally parallel to a vertical plane that bisects a standing wearer into left and right body halves when the article is worn. The transverse axis lies in the plane of the article generally perpendicular to the longitudinal axis. Width is the dimension generally parallel to the transverse axis. Length is the dimension generally parallel to the longitudinal axis. Depth or thickness is the dimension generally parallel to a Z axis perpendicular to the longitudinal and transverse axes.

As used herein, the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241. Meltblown fibers are microfibers, which may be continuous or discontinuous, are generally smaller than 10 microns in average diameter, and are generally tacky when deposited onto a collecting surface.

As used herein, the term “member” when used in the singular can have the dual meaning of a single element or a plurality of elements.

As used herein, the term “nonwoven” and “nonwoven web” refer to materials and webs of material that are formed without the aid of a textile weaving or knitting process.

As used herein, the term “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. The fibers may also have shapes such as those described in U.S. Pat. No. 5,277,976 to Hogle et al., U.S. Pat. Nos. 5,466,410 to Hills and 5,069,970 and 5,057,368 to Largman et al., which describe fibers with unconventional shapes.

As used herein, the term “stretch bonded” refers to an elastic member being bonded to another member while the elastic member is extended at least about 25 percent of its relaxed length. Desirably, the term “stretch bonded” refers to the situation wherein the elastic member is extended at least about 100 percent, and more desirably at least about 300 percent, of its relaxed length when it is bonded to the other member.

As used herein, the term “stretch bonded laminate” refers to a composite material having at least two layers in which one layer is a gatherable layer and the other layer is an elastic layer. The layers are joined together when the elastic layer is in an extended condition so that upon relaxing the layers, the gatherable layer is gathered.

Words of degree, such as “about”, “substantially”, and the like are used herein in the sense of “at, or nearly at, when given the manufacturing, design, and material tolerances inherent in the stated circumstances” and are used to prevent the unscrupulous infringer from unfairly taking advantage of the invention disclosure where exact or absolute figures are stated as an aid to understanding the invention.

### BRIEF DESCRIPTION OF THE DRAWINGS

These and other objects and features of this invention will be better understood from the following detailed description taken in conjunction with the drawings, wherein:

FIG. 1 is a schematic representation of aspects of known methods with regard to leakage control of absorbent disposable garments.

FIG. 2 is a schematic representation of aspects of the present invention with regard to leakage control of absorbent disposable garments.

FIG. 3 representatively shows a perspective view of a disposable garment, according to one embodiment of this invention.

FIG. 4 representatively shows a plan view of a disposable garment in an unfastened, stretched and laid flat condition with a surface of the garment that contacts the wearer's skin facing away from the viewer and portions of the garment partially cut away to more clearly show the interior construction of the garment, according to one embodiment of this invention.

FIG. 5 representatively shows a cross-sectional view of the disposable garment through line V-V as shown in FIG. 4, according to one embodiment of this invention.

FIG. 6 representatively shows a cross-sectional view of the disposable garment according to an alternative embodiment of this invention.

### DESCRIPTION OF PREFERRED EMBODIMENTS

The various aspects and embodiments of the present invention will be described in the context of disposable absorbent articles, and more particularly referred to, without limitation and by way of illustration only, as a disposable diaper. It is apparent that the articles and methods of the present invention are equally adaptable for other types of absorbent articles, such as training pants, adult incontinence articles, feminine care articles, other personal care garments, medical or health care garments, and other disposable articles and garments.

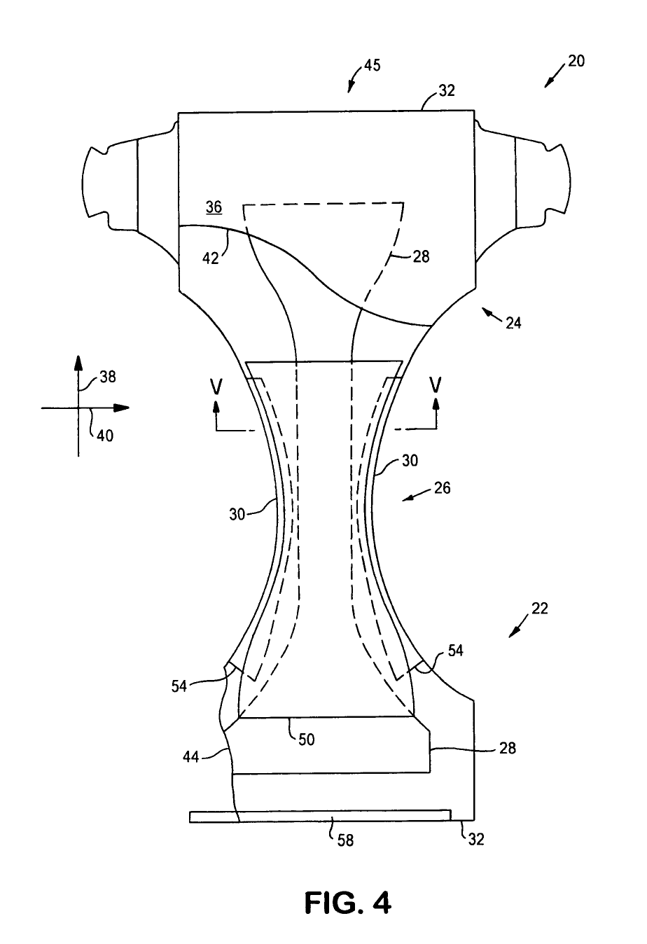

A representative attempt of the known art to prevent any free liquid from exiting within an absorbent garment is seen in FIG. 1, corresponding to FIG. 4 of copending U.S. application Ser. No. 10/245,967, of common ownership herewith. An absorbent core 134 is located between a liner 132 and a backsheet, or outer cover 130. An upper acquisition and distribution layer 152 is located adjacent the liner 132 and a lower acquisition and distribution layer 154 is located adjacent the backsheet 130. A bodily fluid insult, represented by the large arrow 160, contacts the liner 132 generally in the low density, high permeability material of the target area 158 of the retention layer 156 of the absorbent core 134. The fluid of the insult will flow through the absorbent core 134 as indicated by the smaller arrows, collectively 162. The fluid will travel rapidly along the upper acquisition and distribution layer 152 away from the initial point of insult and towards the distal areas 164 of the retention layer 156 in which higher density material having a higher absorption capacity, e.g., a higher concentration of superabsorbent material is located. The fluid will also travel rapidly through the low density, high permeability material of the target area 158 and be carried away from the skin of the wearer located next to the liner 132. The fluid which has traveled through the Z-direction, i.e., thickness, of the retention layer 156 will also be transported away from the target area 158 towards the distal areas 164 by the lower acquisition and distribution layer 154. As per this example, the previous schemes to improve absorption in absorbent garments generally utilize elaborate materials in an attempt to prevent any free liquid from existing within the garment.

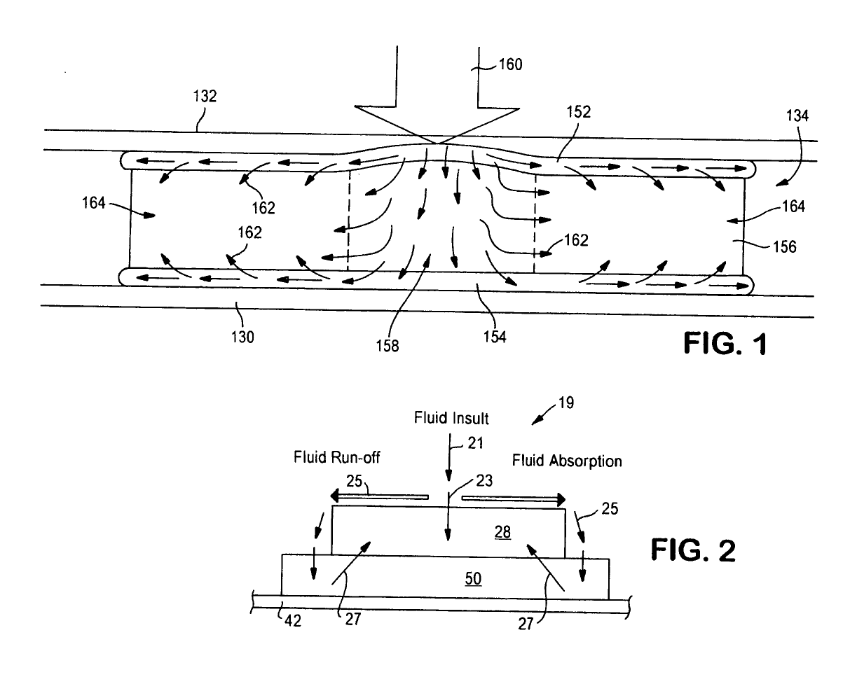

As seen in FIG. 2, an absorbent system 19 of a garment according to aspects of the present invention comprises at least an absorbent core 28 performing the main liquid retention function and a lofty spacer layer 50. A fluid insult 21 is indicated which has an amount or velocity greater than the ability of the absorbent system 19 to immediately capture the liquid, resulting in some indicated absorption 23 and some indicated run off, or free liquid, collectively 25. The run off 25 is captured by the wide lofty spacer layer 50 which is in liquid communication with the absorbent core 28. The run off 25 is then absorbed, as at arrows 27, from the lofty spacer layer 50 by the absorbent core 28 rather than being free to travel along the outer cover 42 of the garment.

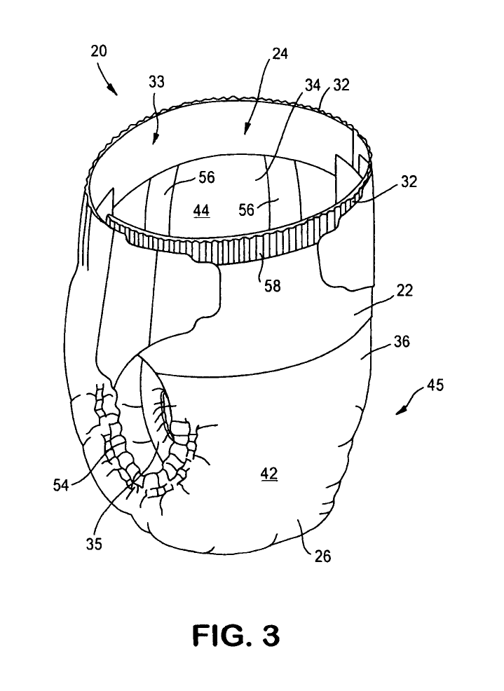

Referring to FIGS. 3-6, in one embodiment of this invention, a diaper 20 comprises a narrow crotch region 26 including a lofty spacer layer 50 having a width in a crotch region 26 of the diaper 20 greater than a width of an absorbent core 28 in the crotch region 26 of the diaper 20. Desirably, the lofty spacer layer 50 has a width in the crotch region 26 at least about 0.25 inches greater than a width of the absorbent core 28 in the corresponding region, more desirably at least about 0.5 inches greater than a width of the absorbent core 28 in the corresponding region, and in some instances may occupy substantially the entire width of the interior of diaper 20 in the crotch region 26. The reduced width of the absorbent core 28 in the crotch region 26 of the diaper 20 provides improved fit while the porous liquid permeable lofty spacer layer 50 provides for distribution of free liquid run off to a second surface of the absorbent core 28 thereby increasing the surface area of the absorbent core 28 available for free liquid absorption, desirably at least doubling the absorption area in the crotch area of the diaper 20.

A disposable diaper 20 of the present invention defines a front waist region 22, a back waist region 24, and an intermediate or crotch region 26 that extends between and connects the front waist region 22 and the back waist region 24. For reference, arrows 38 and 40 depicting the orientation of the longitudinal axis and the transverse or lateral axis, respectively, of the diaper 20 are illustrated in FIG. 4.

The front waist region 22 includes the portion of the diaper 20 that, when worn, is positioned on the front of the wearer while the back waist region 24 includes the portion of the diaper 20 that, when worn, is positioned on the back of the wearer. The crotch region 26 of the diaper 20 includes the portion of the diaper 20 that, when worn, is positioned between the legs of the wearer and covers the lower torso of the wearer. Thus, the crotch region 26 is an area where repeated liquid surges typically occur in the diaper 20 or other disposable absorbent articles.

The diaper 20 defines a pair of laterally opposing side edges 30, a pair of longitudinally opposing waist edges 32, an interior surface 34 that is configured to contact the wearer, and an exterior surface 36 opposite the interior surface 34 that is configured to contact the wearer's clothing during use. Referring to FIGS. 3-6, the diaper 20 also includes an outer cover 42 and a bodyside liner 44 that is connected to the outer cover 42 in a superposed relation. An absorbent core 28 is positioned or located between the outer cover 42 and the bodyside liner 44. The outer cover 42, the bodyside liner 44 and the absorbent core 28 define a chassis 45 of the diaper 20, which forms the waist opening 33 and each leg opening 35. The laterally opposing side edges 30 are generally defined by the side edges of the outer cover 42 that further define leg openings 35, which desirably are curvilinear. The waist edges 32 are generally defined by the waist edges of the outer cover 42 and form the waist opening 33 that is configured to encircle the waist of the wearer during use. The absorbent core 28 is configured to accept and retain body exudates discharged from the wearer.

The diaper 20 may be of various suitable shapes. For example, in the unfastened configuration as shown in FIG. 4, the diaper 20 may have an overall rectangular shape, T-shape or an approximate hourglass shape. In the shown embodiment, the diaper 20 has a generally I-shape in an laid flat, unfastened configuration. A suitable adult incontinence product is described in U.S. patent application Ser. No. 10/261,805, filed Oct. 1, 2002, the disclosure of which is incorporated herein by reference.

The various components of the diaper 20 can be integrally assembled together employing various types of suitable attachment means, such as adhesive, sonic and thermal bonds, and combinations thereof. In the shown embodiment, for example, the outer cover 42 and bodyside liner 44 are assembled to each other and to the absorbent core 28 with an adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, the other components of the diaper 20 may be assembled into the diaper 20 by employing the above-identified attachment mechanisms.

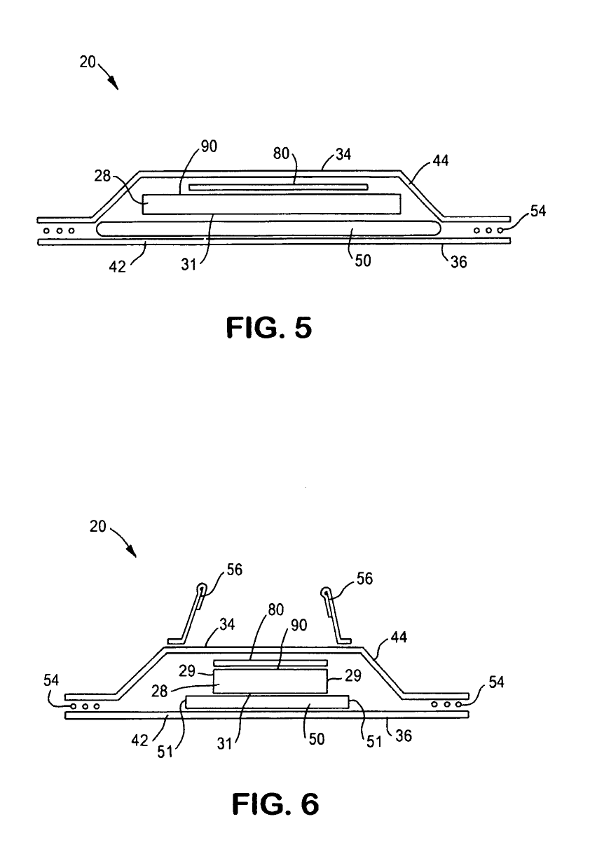

In one embodiment of this invention, e.g., as shown in FIGS. 4 and 5, the outer cover 42 may be extensible, for example as described in U.S. patent application Ser. No. 09/563,417 filed on May 3, 2000 by Roessler et al. The disclosure of application Ser. No. 09/563,417 is incorporated herein by reference. The outer cover 42 of the diaper 20 may suitably be composed of a material that is either water vapor permeable or water vapor impermeable. However, it is generally desired that the outer cover 42 be formed from a material that is substantially impermeable to liquids and permeable to water vapor, i.e., breathable. It is thus accordingly noted that the lofty spacer layer of the present invention will serve as both a fluid distribution layer while still retaining its spacer layer function to allow the outer cover 42 to breath without leaking at higher water vapor transmission values. That is, upon receiving run off liquid, the lofty spacer layer will transmit liquid to the lower surface of the core. The lofty spacer layer will eventually desorb into the core thereby retaining its spacer layer function separating the fluid filled core from the breathable outer cover.

The outer cover 42 can be manufactured from a thin plastic film or other suitable flexible liquid-impermeable material. For example, the outer cover 42 may be formed from a polyethylene film having a thickness of about 0.013 millimeter (0.5 mil) to about 0.051 millimeter (2.0 mils). If a more clothlike feeling is desired, the outer cover 42 may be formed from a polyolefin film having a nonwoven web, such as a spunbond web of polyolefin fibers, laminated to an exterior surface thereof. For example, a stretch-thinned polypropylene film having a thickness of about 0.015 millimeter (0.6 mil) may have thermally laminated thereto a spunbond web of polypropylene fibers. The polypropylene fibers have a thickness of about 1.5 to 2.5 denier per filament, which nonwoven web has a basis weight of about 17 grams per square meter (0.5 ounce per square yard). The outer cover 42 may otherwise include bicomponent fibers such as polyethylene/polypropylene bicomponent fibers. Methods of forming such clothlike outer covers are known to those skilled in the art.

Further, the outer cover 42 may be formed of a woven or nonwoven fibrous web layer which has been totally or partially constructed or treated to impart a desired level of liquid impermeability to selected regions that are adjacent or proximate to the absorbent core 28. Still further, the outer cover 42 may optionally be composed of a microporous “breathable” material, which permits vapors to escape from the absorbent core 28 while still preventing liquid exudates from passing through the outer cover 42. For example, the outer cover 42 may include a vapor permeable nonwoven facing layer laminated to a microporous film. Suitable breathable outer cover materials are described in U.S. Pat. No. 5,695,868, issued to McCormack et al.; and U.S. Pat. No. 5,843,056, issued Dec. 1, 1998 to Good et al., the disclosures of which are incorporated herein by reference. Still further, the outer cover 42 may also be an elastomeric material such as a stretch-thermal laminate (STL), a neck-bonded laminate (NBL), or a stretch-bonded laminate (SBL) material. Methods of making such materials are well known to those skilled in the art and are described in U.S. Pat. No. 4,663,220, issued May 5, 1987 to Wisneski et al.; U.S. Pat. No. 5,226,992, issued Jul. 13, 1993 to Morman; and European Patent Application No. EP 0 217 032 published on Apr. 8, 1987 in the names of Taylor et al., the disclosures of which are incorporated herein by reference. The outer cover 42 can also be embossed or otherwise provided with a matte finish to provide a more aesthetically pleasing appearance.

The bodyside liner 44 suitably presents a body-facing surface that is compliant, soft feeling, and nonirritating to the wearer's skin. In accordance with one embodiment of this invention, the bodyside liner 44 is desirably made of an elastic and/or an extensible material. For example, the bodyside liner 44 may be made from extensible materials as are described in U.S. patent application Ser. No. 09/563,417 filed on May 3, 2000 by Roessler et al. Desirably, these materials are elastic or extensible in at least the transverse or lateral direction, i.e., parallel to the transverse axis depicted by arrow 40 in FIG. 4.

Further, the bodyside liner 44 may be less hydrophilic than the absorbent core 28 to present a relatively dry surface to the wearer and may be sufficiently porous to be liquid permeable, permitting liquid to readily penetrate through its thickness. Suitable web materials for manufacturing the bodyside liner 44 include, but are not limited to, porous foams, reticulated foams, apertured plastic films, natural fibers (for example, wood or cotton fibers), synthetic fibers (for example, polyester or polypropylene fibers), and/or a combination of natural and synthetic fibers. The bodyside liner 44 is suitably employed to help isolate the wearer's skin from liquids held in the absorbent core 28.

Various woven and nonwoven fabrics can be used for the bodyside liner 44. For example, the bodyside liner 44 may be composed of a meltblown or spunbond web of polyolefin fibers. The bodyside liner 44 may also be a bonded-carded web composed of natural and/or synthetic fibers. The bodyside liner 44 may be composed of a substantially hydrophobic material, and the hydrophobic material may optionally be treated with a surfactant or otherwise processed to impart a desired level of wettability and hydrophilicity. In one embodiment of the invention, the bodyside liner 44 is made from a nonwoven, spunbond, polypropylene fabric composed of about 1.5 to about 3.2 denier fibers formed into a web having a basis weight of about 20 grams per square meter and a density of about 0.13 grams per cubic centimeter. The fabric may be surface treated with about 0.3 weight percent of a surfactant commercially available from Hodgson Textile Chemicals, Inc. under the trade designation AHCOVEL Base N-62.

Referencing FIGS. 5 and 6, in one embodiment of this invention, the diaper 20 may, if deemed necessary or desirable, include a surge management layer 80 positioned or located between the bodyside liner 44 and the absorbent core 28 and in liquid communication with the absorbent core 28. For example, the surge management layer 80 may be in liquid communication with a surface 90 of the absorbent core 28. Desirably, the surge management layer 80 comprises a relatively high concentration of fibrous material to temporarily hold the free liquid until the absorbent core 28 is able to absorb the liquid. The surge management layer 80 may also spread the liquid over more surface area of the absorbent core 28 thereby improving distribution of the liquid exudates within the diaper 20 and increasing absorbency efficiency. By providing temporary storage of free liquids, the surge material helps keep the liquid from returning (flowback) through the bodyside liner 44 of the diaper 20, or other absorbent article, and contacting the skin.

The surge management layer 80 is desirably positioned in the crotch region 26 of the diaper 20 and extends longitudinally at least partially into the front waist region 22 of the diaper. In one embodiment of this invention, the surge management layer 80 has a width in the crotch region 26 parallel to the transverse axis 40 less than the width of the absorbent core 28 in the crotch region 26. It is apparent that the surge management layer 80 can have any suitable size dimensions. Examples of suitable surge management layers 80 and particular surge materials are described in U.S. Pat. No. 5,364,382 issued to Latimer; the aforementioned U.S. Pat. No. 5,486,166 issued to Bishop; and U.S. Pat. No. 5,490,846 issued to Ellis, the disclosures of which are incorporated herein by reference. One example of such a surge material could be a through-air bonded carded web composed of 40% 6 dpf (denier per fiber) KoSa T-295 polyester fibers and 60% 1.5-2 dpf KoSa T-258 bicomponent fibers at a basis weight of 3.0 osy having a density of approximately 0.03 g/cc. KoSa, Inc. has offices in Houston, Tex. A surge layer is typically more narrow than the most narrow portion of the absorbent core in the transverse direction of the garment and shorter than the absorbent core in the longitudinal direction of the garment for a known commercial Huggies® Step 3 diaper. Another example of a suitable surge material is a through-air bonded carded web composed of 40% 3 dpf KoSa T-235 or T-121 polyester fiber and 60% 2 dpf KoSa T-256 bicomponent fiber at a basis weight of 3.0 osy having a density of approximately 0.03 g/cc.

The absorbent core 28, such as shown in FIG. 6, may be composed of selected hydrophilic fibers and high-absorbency particles. A most desirable embodiment utilizes a known airformed core which is economical. An airformed core generally consists of natural pulp fibers and superabsorbent particles (SAP) deposited by a process whereby absorbent materials are blown onto a forming drum or wire to form individual batts of unbonded absorbent material suitable for placement in a single garment. The absorbent core 28 of the diaper 20 may suitably be composed of a matrix of hydrophilic fibers, such as a web of cellulosic fluff, mixed with particles of a high-absorbency material commonly known as superabsorbent material. In one embodiment of this invention, the absorbent core 28 includes a matrix of cellulosic fluff such as wood pulp fluff and superabsorbent hydrogel-forming particles.

While the absorbent core is most economically achieved via the airformed core described above, in some cases an airlaid core may be utilized wherein the absorbent core materials include binder materials mixed with the absorbents during forming onto a large collecting form, such as a collecting wire, where the binder materials are then heat activated. The sheets of material are then cut to size for placement in individual garments. The airlaid core construction offers several disadvantages including expense, additional heating steps, and less comfortable construction due to its stiffer nature.

The absorbent core 28 is positioned or located between the bodyside liner 44 and the outer cover 42 to form the diaper 20. The absorbent core 28 desirably has a construction that is generally compressible, conformable, non-irritating to the wearer's skin, and capable of absorbing and retaining body exudates. It should be understood that, for purposes of this invention, the absorbent core 28 may comprise a single, integral piece of material, or alternatively, may comprise a plurality of individual separate pieces of material, which are operably assembled together.

The width of the absorbent core in the crotch region may depend on intended use, for example for baby diapers or adult incontinence products. For example, for baby diapers, the width of the absorbent core 28 generally parallel to the transverse axis 40 in the crotch region 26 desirably is about 2.5 centimeters to about 7.6 centimeters (1.0 inch to about 3.0 inches), more desirably not greater than about 6.4 centimeters (2.5 inches) and even more desirably not greater than about 5.1 centimeters (2.0 inches). For an adult incontinence product of a much larger size, the width of the absorbent core 28 in the crotch region 26 desirably is about 10 centimeters (3.9 inches) to about 25 cm (9.8 in) centimeters, more desirably not greater than about 20 centimeters (7.9 inches), and even more desirably not more than 15 centimeters (5.9 inches). The narrow width of the absorbent core 28 in the crotch region 26 allows the absorbent core 28 to better fit between the legs of the wearer. The absorbent core 28 may have any suitable overall shape. For example, referring to FIG. 6, the absorbent core 28 may be rectangular, I-shaped, or T-shaped. It is apparent that the dimensions and the absorbent capacity of the absorbent core 28 should properly correspond to the size of the intended wearer and the liquid loading imparted by the intended use of the diaper 20. Further, the present invention need not be limited to those cores described, but may indeed be embodied with a variety of open-structure type liquid retention bodies such as foams or the like.

A typical basis weight for absorbent core material in 2.5 inch width crotches can generally be in excess of 1200 gsm in the crotch area, while for a 3.5 inch width crotch, basis weights are generally about 1100 gsm in the crotch region. The different width cores may be at the same density, but would generally have different thicknesses.

Suitable high-absorbency materials for the absorbent core 28 include, but are not limited to, natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of suitable synthetic, polymeric, high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core 28 include, but are not limited to, natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and similar compounds. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention may include FAVOR 880 and FAVOR SXM 9543 polymer available from Stockhausen, Inc., Greensboro, N.C.

The high absorbency material may be in any of a wide variety of geometric forms. Generally, it is desired that the high absorbency material be in the form of discrete particles. However, the high absorbency material may also be in the form of fibers, flakes, rods, spheres, needles, or the like. Generally, the high absorbency material is present in the absorbent core 28 in an amount of about 5 weight percent to about 90 weight percent, based on a total weight of the absorbent core 28.

In some embodiments, a substantially hydrophilic tissue wrapsheet (not shown) may be employed to help maintain the integrity of an airformed fibrous structure of the absorbent core 28. The tissue wrapsheet is typically placed about the absorbent core 28 over at least one of the major facing surfaces thereof and composed of an absorbent cellulosic material, such as creped wadding or a high wet-strength tissue. The tissue wrapsheet may be necked, pleated and/or have any suitable design known to those having ordinary skill in the art to extend in at least one direction, for example the lateral or cross-machine direction, without tearing or ripping.

Referring to FIGS. 4-6, a lofty spacer layer 50 is positioned between the outer cover 42 and the absorbent core 28 and in liquid communication with the absorbent core 28. For example, the lofty spacer layer 50 may be in liquid communication with that surface 31 of the absorbent core 28 distal from the liner 44, as shown in FIGS. 5 and 6. The lofty spacer layer 50 may have any suitable shape consistent with the expected run off patterns of exudate free liquid. The lofty spacer layer 50 is positioned in the crotch region 26 and may extend longitudinally into at least a portion of the front waist region 22 or extend longitudinally into at least a portion of the back waist region 24, or both.

The lofty spacer layer 50 desirably has a width in the crotch region 26 greater than the width of the absorbent layer 28 in the crotch region 26. As shown in FIG. 6, longitudinal edges 51 of the lofty spacer layer 50 extend beyond the longitudinal edges 29 of the absorbent core 28 and may occupy substantially the entire width of the crotch region 26. Desirably, the lofty spacer layer 50 has a width in the crotch region 26 at least about 0.25 inches greater on each side than a corresponding width of the absorbent layer, or core, 28 in the crotch region 26, more desirably at least about 0.5 inches greater. However, the lofty spacer layer 50 may have a width in the front waist region 22 and/or the back waist region 24 equal to or less than the width of the absorbent core 28 if desired.

The lofty spacer layer 50 insulates the outer cover 42 from the absorbent core 28 and provides a porous medium for the passage of run off free liquid to the absorbent core 28. The lofty spacer layer 50 temporarily holds and distributes the run off free liquid to the adjacent surface 31 of the absorbent core 28. As the liquid desorbs from the lofty spacer layer 50 to the core 28, the lofty spacer layer 50 can resume its spacer function of separating the liquid retention function from a breathable outer cover. The porous medium provided by the lofty spacer layer 50 allows the width of the absorbent core 28 in the crotch region 26 to be reduced to a comfortable dimension while providing effective free liquid absorption and management in a diaper 20 of the present invention.

As the width of an absorbent structure, e.g., the surge layer and the absorbent core, in a conventional diaper is reduced, the surface area available for free liquid capture and absorption is reduced. For example, a reduction from about 3.5 inches to about 2.5 inches in the width of a conventional absorbent structure results in a 28% reduction in surface area available for free liquid intake and absorption. Because higher basis weight materials increase the crotch dimension in the thickness or depth axis, wearer comfort may be diminished. As the surface area of a conventional absorbent structure is reduced and the amount of unabsorbed free liquid in the conventional diaper following an insult increases, the possibility of leakage is also increased.

In one embodiment of this invention, the lofty spacer layer 50 comprises a lofty liquid-permeable, resilient material that allows for the passage and distribution of free liquid exudates beneath the absorbent core 28. Suitably, the lofty spacer layer 50 has a basis weight of about 1.5 osy, desirably from about 0.5 to about 3.5, more desirably from about 1.0 to about 2.0 osy, and a density desirably not greater than about 0.1 g/cc, and desirably about 0.25 g/cc to about 0.06 g/cc. The lofty resilient spacer layer 50 may comprise a low density, liquid-porous material such as a 1.5 osy bonded carded web (BCW), composed of 40% 6 dpf (denier per fiber) KoSa T-295 polyester fibers and 60% 1.5-2 dpf KoSa T-258 bicomponent fibers having a web density of approximately 0.03 g/cc.

In some embodiments the lofty resilient spacer layer may be a nonwoven web having a basis weight of about 1.5 osy (51 gsm or grams per square meter) or more, a void volume of between about 60 and about 120 centimeters cubed per gram of web while under a pressure of 0.34 kilopascals (0.05 psi) and a compression resilience in both the wet and dry state of at least about 60 percent. Such a material is generally described in, and measured in accordance with the procedures of, the above-referenced U.S. Pat. No. 5,486,166 to Bishop. Additional suitable materials for the lofty spacer layer 50 include, but are not limited to, a through-air bonded carded web composed of 40% 3 dpf (denier per fiber) KoSa T-235 or T-121 polyester fiber and 60% 2 dpf KoSa T-256 bicomponent fibers at a basis weight of 1.5 osy having a density of approximately 0.03 g/cc. Void volume (cc/g) is dependent upon the material used, and in the case of nonwovens, will be dependent upon fiber size, fiber density and web density. Void volume per area (cc/cm²) is dependent upon the material used and the basis weight of the material. Total void volume (cc) is dependent upon the material used, the basis weight of the material, and the size of the spacer layer in the product.

Current commercially used nonwoven spacer layers of spunbond/meltblown/spunbond (SMS), as used in the experimental examples below, comprise a bonded laminate web of about 0.6 osy, composed of about 37.5% 3.8 dpf spunbond polypropylene fibers, 25% 0.1 dpf meltblown polypropylene fibers, and 37.5% 3.8 dpf spunbond polypropylene fibers, with a density of about 0.067 g/cc, and dimensions of 8.9 cm wide×41.9 cm long for an exemplary Huggies® Step 4 size diaper. These nonwovens exhibit void volume of about 15 cc/g; a void volume per unit area of about 0.03 cc/cm²; and a total void volume of about 11 cc.

A lofty spacer layer, as used in the experimental examples below, of 1.5 osy basis weight, low density (about 0.027 g/cc) through-air bonded carded web composed of 40% 6 dpf Kosa type 295 polyester and 60% 1.5-2 dpf Kosa type 258 bicomponent fiber; and dimensions of 8.9 cm wide×41.9 cm long; exhibit void volume of about 35.5 cc/g; a void volume per unit area of about 0.18 cc/cm²; and a total void volume of about 67 cc.

Desirably, a lofty spacer layer according to the present invention may exhibit void volume of from about 20 cc/g to about 120 or greater cc/g ; in some instances, dependent upon the spacer material selected, desirably above about 20 cc/g; in some instances, desirably above about 30 cc/g; in some instances, desirably above about 35 cc/g.

Desirably, a lofty spacer layer according to the present invention may exhibit a void volume per unit area of from about 0.07 cc/cm² to about 1.0 cc/cm²; in some instances, dependent upon the spacer material type and basis weight selected, desirably above about 0.07 cc/cm²; in some instances, desirably above about 0.15 cc/cm²; in some instances, desirably above about 0.17 cc/cm², in some instances, 0.6 cc/cm² or more.

Desirably, a lofty spacer layer according to the present invention may exhibit a total void volume of from about 23 cc to about 135 cc per product. In some instances, benefit may be had by a smaller lofty spacer occupying less than the full length of the absorbent core. Dependent upon the spacer material type, basis weight, and dimensions selected, the lofty spacer layer has a total void volume desirably above about 30 cc; in some instances, desirably above about 40 cc; in some instances, desirably above about 50 cc; in some instances, desirably above about 60 cc; in some instances, desirably above about 70 cc.

Additional suitable materials for the lofty spacer layer 50 include, but are not limited to open-cell hydrophilic foam, or other materials having similar free liquid handling properties, including any of the above-mentioned surge materials.

Void Volume Test

Thickness of the lofty spacer material was measured using a bulk meter tester in accord with ASTM Standard Test Method for Thickness of Nonwoven Fabrics D 5729-95 using a three inch diameter acrylic platen which provides 0.05 psi (0.34 kilopascals) loading. The sample material should be the same size or larger than the platen.
Void volume for a given platen position is calculated using the equation:

$$VV = \frac{T \cdot {A}_{m} \cdot 0.1 cm \text{/} mm}{M} - \frac{1}{{ρ}_{fiber}}$$

where:
VV=void volume of material in cubic centimeters per gram
T=Thickness of material in millimeters
Aₘ=area of material in square centimeters
M=mass of material in grams
ρ_{fiber}=fiber density in grams per cubic centimeter
   For webs made with multiple fiber types, the web fiber density is the weight average of each individual fiber density:
   ρ_{fiber,Total}=wt %_{fiber 1}·ρ_{fiber 1}+wt %_{fiber 2}·ρ_{fiber 2}.+
   where:
wt %=weight percent of the fiber type in the web or:

$$wt \% = \frac{fiber weight in composition}{total composition weight} \times 100 \%$$

Referring again to FIG. 6, the diaper 20 may further include a pair of containment flaps 56 that are configured to provide a barrier to the lateral flow of body exudates. The containment flaps 56 may be located along the laterally opposing side edges 30 of the diaper 20 adjacent the side edges of the absorbent core 28. Each containment flap 56 typically defines an unattached edge that is configured to maintain an upright, perpendicular configuration in at least the crotch region 26 of the diaper 20 to form a seal against the wearer's body. Each containment flap 56 extends longitudinally along at least a portion of a length of the absorbent core 28. Desirably, each containment flap 56 extends along substantially the entire length of the absorbent core 28 to better contain the body exudates. In accordance with one embodiment of this invention wherein each containment flap 56 extends along a portion of the length of the absorbent core 28, the containment flaps 56 can be selectively positioned along the side edges 30 of the diaper 20 in the crotch region 26. Such containment flaps 56 are generally well known to those skilled in the art. For example, suitable constructions and arrangements for containment flaps 56 are described in U.S. Pat. No. 4,704,116, issued Nov. 3, 1987 to K. Enloe, the disclosure of which is hereby incorporated by reference.

In accordance with one embodiment of this invention, at least a portion of the waist edges 32 and the side edges 30 may be elastic or extensible to improve the fit range of the diaper 20 and support the absorbent core 28 to prevent leakage of body exudates. For example, referring to FIGS. 4-6, the diaper 20 may include a pair of leg elastic members 54 that are connected to the laterally opposing side edges 30 of the diaper 20 in the crotch region 26. The diaper 20 may also include at least one waist elastic member 58 that is connected to a longitudinally opposing waist edge 32 of the diaper 20. The leg elastic members 54 and the waist elastic members 58 are generally adapted to fit about the legs and waist of a wearer, respectively, to maintain a positive, contacting relationship with the wearer to effectively reduce or eliminate the leakage of body exudates from the diaper 20.

### EXAMPLES

Forced Failure Test Procedure

A forced failure test study conducted under confidential conditions was used to evaluate diaper leakage performance. The test procedure involves simulating natural urination in diapers worn by babies in a clinical research setting. The study was conducted with twenty-four babies that used Step 4 Huggies® sized diapers intended for babies weighing 10-17 kg (22-37 lb). Approximately 50% of the babies that participated in this study were female and 50% were male. A gender specific and size appropriate wetting belt was placed on each subject by visually lining up the voiding point of the belt over the urinary meatus of the baby. A test diaper was then applied to the baby by the study conductor. Cotton pants were applied over the test diaper to facilitate leak detection. The test diaper was loaded by pumping 70 ml of 0.9% w/v isotonic NaCl saline solution at a temperature of 35° C. (95° F.) through the wetting belt at a rate of 15 ml/sec. Fluid loading of the test diaper was repeated with a 15-minute interval between loadings until the test diaper leaked and caused a visible area of wetness totaling about 4.9 cm² on the cotton pants. During test diaper fluid loading, 50% of the babies were in the sitting position and 50% in the prone position for both female and male babies. The test diaper was removed from the baby after leakage failure occurred and the amount of fluid it contained was determined by weighing. All test codes in this study were used in a randomized order by all the babies participating in the study.

Code Description

TABLE 1
study matrix

| | | Lofty Spacer | SMS Spacer |
|---|---|---|---|
| Absorbent Core | High | A | I |
| Type | Permeability | | |
| | Low | B | J |
| | Permeability | | |

Chassis

A Huggies® Step 4 sized diaper chassis design with a T-shaped absorbent pad having a 64 mm (2.5 inch) wide crotch at its narrowest dimension was used. All codes in this study used the same chassis type and construction except for the spacer layers and absorbent cores as indicated.

Spacer Layer

Codes A& B used a lofty spacer layer of 1.5 osy basis weight, low density (about 0.027g/cc) through-air bonded carded web composed of 40% 6 dpf Kosa type 295 polyester and 60% 1.5-2 dpf Kosa type 258 bicomponent fiber. Dimensions were 8.9 cm wide×41.9 cm long. Void volume of this lofty spacer layer is about 35.5 centimeters cubed per gram.

Codes I & J used a known, commercially used, spunbond/meltblown/spunbond (SMS) spacer layer, a higher density (about 0.067 g/cc) bonded laminate web of about 0.6 osy composed of about 37.5% 3.8 dpf spunbond polypropylene fibers, 25% 0.1 dpf meltblown polypropylene fibers, and 37.5% 3.8 dpf spunbond polypropylene fibers. Dimensions were 8.9 cm wide×41.9 cm long. Void volume of this SMS spacer is about 15 centimeters cubed per gram.

Absorbent Core:

Two types of absorbent cores were used. One was a thermally stabilized airlaid and the other an unstabilized airformed absorbent. Due to binder fiber stabilization, the airlaid material better resists the wet structural collapse that occurs with unstabilized airformed materials. As a result, airlaids maintain higher fluid permeability resulting in superior fluid intake functionality compared to airformed materials.

The absorbent core used in codes A & I, designated “High permeability” (higher intake rate) was a thermally stabilized airlaid material that comprised 50% SAP, about 46% Weyerhaeuser NB416 pulp and about 4% Kosa T255 bicomponent binder fibers. The basis weight of the absorbent core in the front target zone region was about 1150 gsm and about 420 gsm in the non-zoned back portion of the absorbent. The absorbent density was about 0.2 g/cc.

The absorbent core used in codes B & J, designated “Low permeability” (lower intake rate) was an unstabilized airformed material consisting of about 43% SAP and 57% Bowater CR1654 pulp. The basis weight of the absorbent core in the front target zone region was about 1150 gsm and about 380 gsm in the non-zoned back portion of the absorbent. The absorbent density was about 0.2 g/cc.

Table 2 contains the mean load at failure, and the statistical grouping information based on the mean load at failure data.

TABLE 2
Statistical results at 90% confidence

| | | Mean Load |
|---|---|---|
| Code | Description | at Leak (g) |
| A | High permeability absorbent, lofty spacer | 365 A |
| B | Low permeability absorbent, lofty spacer | 352 A |
| I | High permeability absorbent, SMS spacer | 343 A |
| J | Low permeability absorbent, SMS spacer | 263 B |

The lofty spacer layer provided a significant leakage benefit with low permeability absorbent cores in mean load at leak. The difference between the mean load at failure for codes B and J was highly significant at the 99% Confidence level, meaning that this difference was not likely due just to chance.

The lofty spacer layer provided improvement for higher permeability absorbent cores for mean load at leak in a non-statistical sense that, at face value, the High permeability absorbent cores with a lofty spacer layer had a higher mean load at failure than the High permeability absorbent cores without the lofty spacer layer. The difference between the mean load at failure for codes A and I was not strictly statistically significant.

The benefit of the lofty spacer layer is more apparent in lower permeability absorbent structures due to the higher incident of run off thereby allowing the lofty spacer mechanism to work. This benefit is reduced in the higher permeability absorbent cores due to the reduced potential for run off. However, it can generally be seen that by using a lofty spacer layer, an airformed absorbent core diaper can be made to perform as well as a more expensive stabilized airlaid absorbent core utilizing a current thin spacer layer in the diaper, at lower material cost, capital cost, higher efficiency, and reduced process complexity. Also, by using a lofty spacer, an airformed absorbent core diaper can perform as well as a stabilized airlaid absorbent core diaper without the adverse effect on fit and comfort of reduced absorbent flexibility and conformability associated with stabilized airlaid material.

A similarly confidential forced failure study showed still another benefit of the present invention in that a narrower crotch absorbent core can be made to perform as well as a wider crotch absorbent core and therefore the garment may provide improved fit without a tradeoff in leakage performance. Table 3 shows that, with all other things being equal, a diaper utilizing a lofty spacer layer and a narrower absorbent core combination according to the present invention can exhibit nearly equal performance to that of a wider crotch area absorbent core that does not utilize a lofty spacer layer.

TABLE 3
Forced Failure Test-Step 4 Huggies ® size chassis

| | | Spacer Width | Mean Load at |
|---|---|---|---|
| Crotch Width | Spacer Material | (mm) | Leak(g) |
| 2.5 inch (64 mm) | 0.7 osy SMS | 89 | 304 B |
| 2.5 inch (64 mm) | 1.5 osy Lofty spacer | 89 | 325 A |
| 3.0 inch (76 mm) | 0.7 osy SMS | 89 | 330 A |

While in the foregoing specification this invention has been described in relation to certain exemplary embodiments or aspects thereof, and many details have been set forth for purpose of illustration, it will be apparent to those skilled in the art that the invention is susceptible to additional embodiments and that certain of the details described herein can be varied considerably without departing from the basic principles of the invention.

1. A disposable absorbent article having a front waist region, a back waist region and a crotch region intermediate the front waist region and the back waist region, consisting essentially of:
a body-side liner for facing the wearer;
an outer cover,
an absorbent core having a first surface facing the liner and a second surface facing the outer cover,
a lofty spacer layer positioned between the absorbent core and the outer cover, and in liquid communication with at least a portion of the second surface of the absorbent core in the crotch region;
the lofty spacer layer having a void volume of 20 centimeters cubed per gram of web or higher under a pressure of 0.34 kilopascals.

2. A disposable absorbent article of claim 1 wherein the lofty spacer layer has a void volume of between about 30 and about 120 centimeters cubed per gram of web while under a pressure of 0.34 kilopascals.

3. A disposable absorbent article of claim 1 wherein the lofty spacer layer has a void volume of above 30 centimeters cubed per gram of web while under a pressure of 0.34 kilopascals.

4. A disposable absorbent article of claim 1 wherein the lofty spacer layer has a void volume of above 40 centimeters cubed per gram of web while under a pressure of 0.34 kilopascals.

5. A disposable absorbent article of claim 1 wherein the lofty spacer layer has a void volume of above 50 centimeters cubed per gram of web while under a pressure of 0.34 kilopascals.

6. A disposable absorbent article of claim 1 wherein the lofty spacer layer has a void volume of above 60 centimeters cubed per gram of web while under a pressure of 0.34 kilopascals.

7. A disposable absorbent article of claim 1 wherein the lofty spacer layer has a void volume of above 70 centimeters cubed per gram of web while under a pressure of 0.34 kilopascals.

8. A disposable absorbent article of claim 1 wherein the lofty spacer layer has a total void volume of at least 23 cubic centimeters.

9. A disposable absorbent article of claim 1 wherein the lofty spacer layer has a total void volume of at least 30 cubic centimeters.

10. A disposable absorbent article of claim 1 wherein the lofty spacer layer has a total void volume of at least 40 cubic centimeters.

11. A disposable absorbent article of claim 1 wherein the lofty spacer layer has a total void volume of at least 50 cubic centimeters.

12. A disposable absorbent article of claim 1 wherein the lofty spacer layer has a total void volume of at least 60 cubic centimeters.

13. A disposable absorbent article of claim 1 wherein the lofty spacer layer has a total void volume of at least 70 cubic centimeters.

14. The disposable absorbent article of claim 1 wherein the lofty spacer layer has a void volume per surface area of between about 0.07 to about 1.0 cubic centimeters per square centimeter.

15. The disposable absorbent article of claim 1 wherein the lofty spacer layer has a void volume per surface area of at least 0.07 cubic centimeters per square centimeter.

16. The disposable absorbent article of claim 1 wherein the lofty spacer layer has a void volume per surface area of at least 0.15 cubic centimeters per square centimeter.

17. The disposable absorbent article of claim 1 wherein the lofty spacer layer has a void volume per surface area of at least 0.17 cubic centimeters per square centimeter.

18. A disposable absorbent article of claim 1 further consisting essentially of a surge management layer positioned above a first surface of the absorbent core, and in liquid communication with a first surface of the absorbent core.

19. The disposable absorbent article of claim 1 wherein the lofty spacer layer extends beyond each lateral edge of the absorbent core by at least 0.25 inches in the narrowest part of the absorbent core.

20. The disposable absorbent article of claim 1 wherein the lofty spacer layer extends beyond each lateral edge of the absorbent core by at least 0.5 inches in the narrowest part of the absorbent core.

21. The disposable absorbent article of claim 1 wherein the lofty spacer layer has a permeability greater than the absorbent core.

22. The disposable absorbent article of claim 1 wherein the spacer layer has a basis weight of about 0.5 osy or greater.

23. The disposable absorbent article of claim 1 wherein the spacer layer has a basis weight of about 1.0 osy or greater.

24. The disposable absorbent article of claim 1 wherein the spacer layer has a basis weight of about 1.5 osy or greater.

25. The disposable absorbent article of claim 1 wherein the spacer layer has a basis weight between about 0.5 and about 3.5 osy.

26. The disposable absorbent article of claim 1 wherein the width of the absorbent core in the crotch region is about 1.5 inches to about 2.5 inches.

27. The disposable absorbent article of claim 26 wherein the absorbent article is a diaper, training pant or swim pant.

28. A disposable absorbent article of claim 1 for use on an adult wherein the width of the absorbent core in the crotch region is about 3.9 to about 7.9 inches.

29. The disposable absorbent article of claim 1 wherein the outer cover is breathable.

30. The disposable absorbent article of claim 1 wherein the outer cover is elastic or extensible.

31. The disposable absorbent article of claim 1 wherein the absorbent core has a permeability permitting free liquid run off from a first surface thereof when exposed to a liquid insult on the first surface.

32. The disposable absorbent article of claim 1 wherein the absorbent core is airformed.

33. The disposable absorbent article of claim 32 wherein the absorbent core consists essentially of superabsorbent material and pulp material.

34. The disposable absorbent article of claim 32 wherein the absorbent core consists essentially of a matrix of hydrophilic fibers and high-absorbency materials.

35. The disposable absorbent article of claim 1 wherein the lofty spacer layer is a nonwoven web having a compression resilience in both the wet and dry state of at least about 60 percent.

36. The disposable absorbent article of claim 1 wherein the lofty spacer layer has a density of less than 0.060 g/cc.

37. The disposable absorbent article of claim 1 further consisting essentially of a surge layer positioned between the wearer and the absorbent core, and in liquid communication with the second surface of the absorbent core.

38. A disposable absorbent article comprising:
an outer cover;
a bodyside liner at least partially bonded to the outer cover in a superposed relation;
an absorbent core positioned between the outer cover and the bodyside liner, the outer cover, the bodyside liner and the absorbent core defining a chassis having a front waist region, a back waist region and a crotch region intermediate the front waist region and the back waist region for covering a wearer, the absorbent core having a width in the crotch region not greater than about 3.0 inches;
the absorbent core comprising an airformed matrix of hydrophilic fibers and high-absorbency materials
a surge layer positioned between the bodyside liner and the absorbent core, and in liquid communication with a first surface of the absorbent core; and
a liquid permeable lofty spacer layer positioned between the absorbent core and the outer cover, the liquid permeable lofty spacer layer in liquid communication with at least a portion of the second surface of the absorbent core, the liquid permeable lofty spacer layer having a void volume of above about 20 centimeters cubed per gram of web while under a pressure of 0.34 kilopascals, the lofty spacer layer having a basis weight of between about 0.5 osy to about 3.5 osy.

39. The disposable absorbent article of claim 38 wherein the outer cover is extensible or elastic.

40. The disposable absorbent article of claim 38 further comprising the liquid permeable lofty spacer layer having a compression resilience in both the wet and dry state of at least about 60 percent.

41. The disposable absorbent article of claim 38 further comprising the liquid permeable lofty spacer layer having a void volume per surface area of about 0.07 to about 1.0 centimeters cubed per square centimeter of web.

42. The disposable absorbent article of claim 38 further comprising the liquid permeable lofty spacer layer having a void volume per surface area of at least 0.07 centimeters cubed per square centimeter of web.

43. The disposable absorbent article of claim 38 further comprising the liquid permeable lofty spacer layer having a total void volume of about 65 cc.

44. The disposable absorbent article of claim 41 further comprising the liquid permeable lofty spacer layer having a width in the crotch region at least about 0.50 inches greater than the width of the absorbent core in the crotch region.

45. The disposable absorbent article of claim 41 wherein the width of the absorbent core in the crotch region is about 1.5 inches to about 2.5 inches.

46. The disposable absorbent article of claim 45 wherein the width of the lofty spacer layer in the crotch region is about 2.00 inches to about 4.00 inches.

47. A disposable absorbent article having a front waist region, a back waist region and a crotch region intermediate the front waist region and the back waist region, consisting essentially of:
a body-side liner for facing the wearer;
an outer cover,
an absorbent core having a first surface facing the liner and a second surface facing the outer cover,
a lofty spacer layer positioned between the absorbent core and the outer cover, and in liquid communication with at least a portion of the second surface of the absorbent core in the crotch region;
the lofty spacer layer having a total void volume of at least 23 cubic centimeters under a pressure of 0.34 kilopascals.

48. A disposable absorbent article of claim 47 wherein the lofty spacer layer has a total void volume of at least 30 cubic centimeters.

49. A disposable absorbent article of claim 47 wherein the lofty spacer layer has a total void volume of at least 40 cubic centimeters.

50. A disposable absorbent article of claim 47 wherein the lofty spacer layer has a total void volume of at least 50 cubic centimeters.

51. A disposable absorbent article of claim 47 wherein the lofty spacer layer has a total void volume of at least 60 cubic centimeters.

52. A disposable absorbent article of claim 47 wherein the lofty spacer layer has a total void volume of at least 70 cubic centimeters.

53. A disposable absorbent article of claim 47 wherein the lofty spacer layer has a void volume of at least 20 cubic centimeters per gram of web while under a pressure of 0.34 kilopascals.

54. A disposable absorbent article of claim 47 wherein the lofty spacer layer has a void volume of at least 30 cubic centimeters per gram of web while under a pressure of 0.34 kilopascals.

55. A disposable absorbent article of claim 47 wherein the lofty spacer layer has a void volume of at least 35 cubic centimeters per gram of web while under a pressure of 0.34 kilopascals.
